(19) United States
(12) Patent Application Publication
(10) Pub. No.: US 2020/0054746 A1
(43) Pub. Date: Feb. 20, 2020

(54) METHODS FOR INCREASING NEURONAL SURVIVAL

(71) Applicant: The General Hospital Corporation, Boston, MA (US)

(72) Inventors: Mark William Albers, Winchester, MA (US); Luxiang Cao, Andover, MA (US)

(21) Appl. No.: 16/663,038

(22) Filed: Oct. 24, 2019

Related U.S. Application Data
(63) Continuation of application No. 15/025,648, filed on Mar. 29, 2016.
(60) Provisional application No. 61/886,390, filed on Oct. 3, 2013.

(51) Int. Cl. A61K 39/395; A61K 31/704; A61K 31/366; A61K 31/351; A61K 31/713; A61K 45/06

(52) U.S. Cl. CPC A61K 39/3955; A61K 31/704; A61K 31/366; A61K 2039/505; A61K 31/713; A61K 45/06; A61K 31/351

(57) ABSTRACT

Described are methods of treating a subject after acute trauma to the head, viral encephalitis, or other causes of acute neurodegeneration, and/or after acute vascular insults, including ischemic and hemorrhagic strokes, the method comprising administering inhibitors of BACE1; an inhibitory oligonucleotide targeting BACE1; and/or an inhibitory oligonucleotide targeting APLP2. Also described are methods for treating a subject who is at high risk for head trauma, including administering a prophylactically effective amount (i.e., an amount sufficient to reduce the risk of developing or reduce the severity or duration of symptoms of head trauma) of one or more of an inhibitor of BACE1; an inhibitory oligonucleotide targeting BACE1; or an inhibitory oligonucleotide targeting APLP2.

### CLAIM OF PRIORITY

This application is a continuation of U.S. patent application Ser. No. 15/025,648, filed Mar. 29, 2016, which is a U.S. National Phase Application under 35 U.S.C. § 371 of International Patent Application No. PCT/US2014/059018, filed on Oct. 3, 2014, which claims the benefit of U.S. Patent Application Ser. No. 61/886,390, filed on Oct. 3, 2013. The entire contents of the foregoing are hereby incorporated by reference.

### FEDERALLY SPONSORED RESEARCH OR DEVELOPMENT

This invention was made with Government support under Grant No. NIH DP2-OD006662 awarded by the National Institutes of Health. The Government has certain rights in the invention.

### TECHNICAL FIELD

Described are methods of treating a subject after acute trauma to the head, viral encephalitis, or other causes of acute neurodegeneration, and/or after acute vascular insults, including ischemic and hemorrhagic strokes, or for treating a subject who is at high risk for head trauma. The methods include administering inhibitors of BACE1; an inhibitory oligonucleotide targeting BACE1; and/or an inhibitory oligonucleotide targeting APLP2.

### BACKGROUND

An estimated 1.4 million Americans are affected by traumatic brain injury (TBI) each year. It is the largest cause of the disability among people under the age of 45 and has a devastating economic impact (1). Developing therapies for TBI is important to augment the quality of life of its victims, and to reduce its economic toll to society. A wealth of basic and clinical research indicates two phases underlie the dysfunctions observed clinically (2). The primary injury is the result of the mechanical disruption at the time of exposure of the external force and triggers a second phase of molecular cascades and cellular events that evolve over the ensuing minutes to months and ultimately lead to brain cell death (3, 4). Prevention is the primary therapeutic modality for the primary phase, whereas pharmacologic manipulation of these molecular cascades and cellular events, including cell death (5-7), has been the therapeutic goal for the second phase (8). In animal models, numerous pathways have been identified, and therapeutic interventions have been successful. Unfortunately, the translation of these findings in animal models to therapies in humans has not been successful to date, despite multiple clinical trials (8). Several reasons have been suggested for these discrepancies, including animal models that may not faithfully reproduce the injury in humans, lack of adequate pharmacokinetics to ensure that the drug achieved therapeutic levels in human brains, and interference of the restoration process in spite of adequate neuroprotection (3).

Anosmia is a common debilitating consequence of TBI, with a profound impact on the quality of life (9-13). Deficits of odor discrimination, odor detection threshold, and odor identification have been reported in individuals following head trauma at a variety of time points past injury (12). Patients with partial or complete anosmia are at greater risk of depression (14) and danger from environmental hazards, e.g. spoiled foods, fire, and natural gas leaks (12, 15). Therapeutic agents to aid the recovery of olfactory function have been largely anecdotal, e.g., theophylline, and not subject to rigorous placebo-controlled clinical trials (16). The loss of smell function is often due to injury of axons of primary olfactory sensory neurons (OSNs) as they pass through the cribiform plate of the skull from the nose to the brain. The axonal injury is thought to provoke increased calcium signaling, caspase activation, and often leads to death of neurons (5). Since OSNs are self-renewing, an injury augments neurogenesis in the olfactory epithelium. Moreover, the receptivity of second order olfactory neurons to integrate new axons into existing neural circuits is essential to restoring function (17, 18), which affords simultaneous evaluation of neuroprotective efficacy without interfering with endogenous neurorestorative processes. Ultimately, therapies developed for olfactory neurons following acute injury will not only alleviate anosmia, but also may be applicable to other neurons in the central nervous system.

### SUMMARY

The present invention is based, at least in part, on the development of a reliable, reproducible paradigm of surgical injury to axons of olfactory sensory neurons (OSNs) after they pass through the cribiform plate into the brain, but prior to innervating the olfactory bulb and establishing synapses, modeling the shear injury and diffuse axonal injury observed in humans. Unilateral OSN axotomy results in robust axonal cleaved caspase 3 activation and retrograde propagation of activated caspase 3 and cell death of the majority of mature OSNs in affected regions. This massive OSN degeneration and epithelial thinning observed 3 days after acute axonal injury is largely prevented (˜50% preservation of OSNs) in BACE1+/− mice in spite of robust induction of cleaved caspase 3. Surprisingly, OSN loss is slightly exacerbated in BACE1-null mice, perhaps reflecting the differential biological effects of numerous BACE1 substrates involved in intracellular trafficking in the setting of acute axonal injury. In addition, a similar degree of neuroprotection of olfactory neurons was observed in the same paradigm in APLP2-null mice. APLP2 is a validated substrate cleaved by the BACE1 protease. Together, these data demonstrate that BACE1-dependent cleavage is a critical event in the complex molecular cascade linking axonal injury to neuronal death and that APLP2 plays an essential role in mediating neuronal death after axonal injury.

Thus, in a first aspect, the invention provides methods for treating a subject after acute trauma to the head, viral encephalitis, or other causes of acute neurodegeneration, and/or after acute vascular insults, including ischemic and hemorrhagic strokes. The methods include administering a therapeutically effective amount of one or more of: an inhibitor of BACE1; an inhibitory oligonucleotide targeting BACE1; or an inhibitory oligonucleotide targeting APLP2.

In another aspect, the invention provides methods for treating a subject who is at high risk for head trauma, e.g., an athlete or soldier. The methods include administering a prophylactically effective amount (i.e., an amount sufficient to reduce the risk of developing or reduce the severity or duration of symptoms of head trauma) of one or more of: an inhibitor of BACE1; an inhibitory oligonucleotide targeting BACE1; or an inhibitory oligonucleotide targeting APLP2.

In another aspect, the invention provides methods for treating a chronic neurodegenerative disease in a subject, e.g., Alzheimer's disease, Parkinson's disease, Huntington's disease, or frontotemporal dementia. The methods include administering a therapeutically effective amount of an inhibitory oligonucleotide targeting APLP2.

In some embodiments, the treatment promotes survival of neurons in the central nervous system.

In some embodiments, the treatment promotes survival of olfactory sensory neurons.

In some embodiments, the inhibitor of BACE1 is a small molecule or antibody that binds to BACE1.

In some embodiments, the inhibitor of BACE1 is selected from the group consisting of LY2886721 and LY2811376 (Lilly); MBI-1, MBI-3, MBI-5, and MK-8931 (Merck); E2609 (Eisai); RG7129 (Roche); TAK-070 (Takeda); CTS-21166 (CoMentis); AZD3293 and AZ4217 (AstraZeneca); HPP854 (High Point Pharmaceuticals); Ginsenoside Rg1 (CID 441923); Hispidin (CID310013); TDC (CID 5811533); Monacolin K (CID 53232); PF-05297909; SCH 1359113; Spirocyclic inhibitors (e.g., compound (R)-50); fluorine-substituted 1,3-oxazines (e.g., the CF3 substituted oxazine 89).

In some embodiments, the inhibitor of BACE1 is a bispecific antibody, e.g., with one arm targeting BACE and the other recognizing transferrin receptor to boost brain penetrance, or a camelid antibody that bind and inhibit BACE1.

In some embodiments, the oligonucleotide is 15 to 21 nucleotides in length.

In some embodiments, at least one nucleotide of the oligonucleotide is a nucleotide analogue.

In some embodiments, at least one nucleotide of the oligonucleotide comprises a 2′ O-methyl.

In some embodiments, the oligonucleotide comprises at least one ribonucleotide, at least one deoxyribonucleotide, or at least one bridged nucleotide.

In some embodiments, the bridged nucleotide is a LNA nucleotide, a cEt nucleotide or a ENA modified nucleotide.

In some embodiments, each nucleotide of the oligonucleotide is a LNA nucleotide.

In some embodiments, one or more of the nucleotides of the oligonucleotide comprise 2′-fluoro-deoxyribonucleotides, 2′-O-methyl nucleotides, ENA nucleotide analogues, or LNA nucleotides.

In some embodiments, the nucleotides of the oligonucleotide comprise phosphorothioate internucleotide linkages between at least two nucleotides.

In some embodiments, the oligonucleotide is a gapmer or a mixmer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F: Axotomy of OSNs emerging through cribiform plate results in cleaved caspase 3 induction, retrograde transport of cleaved caspase 3, and neuronal death. (A) Schematic diagram of axotomy injury model illustrating the location of the loop (thin wire blade) insertion caudal to the cribiform plate between the skull and olfactory bulb. (B-E) Representative images of dorsal olfactory epithelium sections rostral to the cribiform plate of injured and non-injured sides after immunostaining with an antibody against cleaved caspase 3 illustrating retrograde transport of cleaved caspase 3 into axon bundles (black signal in B). The loss of thickness of the epithelium (indicated by the less nuclei stained by DAPI in C and the white double arrows in D and E to illustrate the loss of OSNs at 3 d post injury. (F) Quantification of the epithelial thickness in affected regions at 3 d, 7 d, and 14 d after injury. Note regeneration of OSNs at 7d and 14 d post injury.(mean±s.e.m.).

FIG. 2: Loss of mouse APP or BACE1 do not confer neuroprotection following acute axonal injury. Axotomy of olfactory sensory neurons in mice with constitutive knockout of APP, which is necessary for the production for the Aβ peptide, does not offer neuroprotection 3 d after the injury. Axotomy of olfactory neurons in mice with constitutive knockout of BACE1, which is necessary for the production for the Aβ peptide, does not offer neuroprotection 3 d after the injury. Quantification of epithelial thickness in wild type, APP, and BACE1 knockout lines.

FIGS. 3A-C. Reduction of Cell Apoptosis in APLP2−/− Mice. (A) Schematic drawing of TUNEL-positive olfactory sensory neurons (OSNs) in one half of an epithelium of a wild type mouse. (B) Schematic drawing of TUNEL-positive OSNs in epithelium of an APLP2−/− mouse. (C) Quantification of the density of TUNEL-positive OSNs (# of neurons/total epithelial area). Density in APLP2−/− is normalized to the density of littermate controls (n=4 pairs; p<0.003, one-way ANOVA).

FIGS. 4A-B. Decreased Neurogenesis and Increased OSN Half Life in APLP2−/− Mice. (A) Quantification of normalized density of BrDU+ OSNs in WT and APLP2−/− mice 2 hours after BrDu injection(n=5). (B) Quantification of normalized density of BrDU+ OSNs in WT and APLP2−/− mice, 30 days after injection (n=4).

FIG. 5: APLP2 Deficiency Rescues Axotomy Induced Neurodegeneration. Quantificationof normalized epithelial thickness of injured side to the untreated side, 3 d (n=5), 7 d (n=5), and 14 d (n=5) in APLP2−/− mice. In APLP2−/− mice, the loss of olfactory epithelial thickness is only half that seen in the wild type mouse (FIG. 1). The epithelial thickness is not significantly different between the 3 d, 7 d and 14 d samples, indicating that these neurons are resistant to the injury in spite of robust induction of cleaved caspase 3. Immunohistochemistry revealed that increased thickness is due to an increased population of mature OSNs.

FIG. 6: BACE1 Partial Deficiency Rescues Axotomy Induced Neurodegeneration. Comparison of normalized epithelial thickness of injured side of wild type (blue) or BACE1+/− (green) mice at each time point. N≥3 for each time point. Immunohistochemistry reveals that increased thickness is due to an increased population of mature OSNs.

FIG. 7: BACE1−/− Deficiency Does Not Rescue Axotomy-Induced OSN Neuronal Loss and Epithelial Thinning. Comparison of normalized epithelial thickness of injured side of wild type (blue) or BACE1+/− (green) and BACE1−/− mice at 3 days reveals significant protection in the BACE1 heterozygous mouse (p<0.02 relative to wild type) and exacerbation in the BACE1 null mouse (p<0.02 relative to wild type mice). N≥3 for each time point. Immunohistochemistry reveals that increased thickness is due to an increased population of mature OSNs.

### DETAILED DESCRIPTION

Olfactory sensory neurons are exposed to oxidative stress and toxins in the environment and undergo constant degeneration and replenishment via neurogenesis. One of the markers of diffuse axonal injury (19) is elevated levels of the amyloid precursor protein (APP) and the beta-APP cleaving enzyme 1 (BACE1), resulting in increased production of the Aβ peptide (20). Both APP and Aβ have been implicated in Alzheimer's disease (AD); specific expression of an isoform of human APP increases Aβ production, and misexpression of the human Aβ40 or human Aβ42 peptides alone are sufficient to cause olfactory deficits in mice (17).

Described herein is a unique role for the APLP2 protein, a homolog of APP, to regulate physiological neuronal turnover, which may contribute to age-associated loss of neurological and cognitive function. Moreover, we discovered that APLP2 modulates the active program that mediates injury-induced neuronal cell death in olfactory sensory neurons. Together, these findings implicate the APLP2 gene and gene products as targets to reduce neuronal death after acute injuries, such as head trauma, an ischemic vascular event, or hemorrhage, thereby promoting an improved functional outcome. Moreover, in adult mice, inhibition of expression of the BACE1 protease, a validated cleavage enzyme of APLP2, also protects OSNs from neuronal death following deafferentation. These results also support the BACE1 protease as a target to reduce neuronal death after acute injury to neurons, especially to axons that project within the central nervous system. The invention includes both acute (immediately after injury) therapeutic interventions and chronic preventive strategies for individuals at risk for head trauma (e.g., athletes and soldiers). The concerns related to interfering with axon targeting on nascent neurons (30, 31) are alleviated with the recent description of a critical period for olfactory axons (32, 33) and full recovery of the intact map after a loss of >99% of OSNs in adult mice (32).

Thus, the present methods include the administration of BACE1 inhibitors, e.g., BACE1 protease inhibitors that can penetrate the blood brain barrier, and/or inhibitory oligonucleotides (“oligos”) that specifically reduce APLP2 and BACE1 gene product expression.

These agents can be used therapeutically, e.g., after injury, or prophylactically, e.g., before injury in a subject who has a high risk of sustaining a head injury (e.g., an athlete during the sporting season, or a soldier going into combat).

Therapeutic administration, preferably administered acutely after injury, e.g., commencing within 48, 24, 18, 12, 6, 4 or 2 hours after injury, will promote protection of neurons in the central nervous system, including prolonged survival, after acute trauma to the head, viral encephalitis, or other causes of acute neurodegeneration; and/or promote protection of neurons in the central nervous system, including prolonged survival, e.g., after acute vascular insults, including ischemic and hemorrhagic strokes.

Prospective or prophylactic administration of BACE1 inhibitors to individuals at high risk for head trauma (athletes during the season, and soldiers going into combat) will promote protection of neurons in the central nervous system, including prolonged survival, after acute trauma to the head. Prospective or prophylactic administration of inhibitory nucleic acids that specifically reduce APLP2 gene product expression will promote protection of neurons in the central nervous system, including prolonged survival, in patients at risk of developing or with evidence of a chronic neurodegenerative disease (e.g., an early, preclinical stage of the disease where biomarkers of the disease suggest the presence of the disease process; biomarkers could include but are not limited to olfactory dysfunction, changes in the cerebral spinal fluid, specific volumetric loss of brain tissue revealed by magnetic resonance imaging, diminished metabolism, decreased synaptic density, or aggregation of pathologic protein by positron emission tomography), including but not limited to Alzheimer's disease, Parkinson's disease, Huntington's disease, and frontotemporal dementia.

In some embodiments, the methods are used to treat a subject who has had a traumatic brain injury (TBI) or spinal injury, e.g., caused by mechanical force or as a result of mechanical force. In these subjects, treatment using the methods described herein can result in an improvement (e.g., return to or towards normal) in one or more clinical parameters, e.g., reduced duration of loss of consciousness; reduced posttraumatic amnesia; or functioning as measured by the Glasgow Coma Scale (GCS), Functional Independence Measure (FIM), or Disability Rating Scale (DRS); or complications of TBI including insomnia, cognitive decline, posttraumatic headache, posttraumatic depression, posttraumatic seizures, hydrocephalus, deep vein thrombosis, heterotopic ossification, spasticity, gastrointestinal and genitourinary complications, gait abnormalities, or agitation.

In some embodiments, the methods are used to treat a subject who has had an injury to their olfactory nerve (cranial nerve I), e.g., associated with TBI. In these subjects, treatment using the methods described herein can result in an improvement (e.g., return to or towards normal) in symptoms, e.g., in one or more of anosmia (loss of the sense of smell), hyposmia (a decreased sense of smell), parosmia (a perversion of the sense of smell), or cacosmia (awareness of a disagreeable or offensive odor that does not exist).

In some embodiments, the methods can include evaluating and monitoring the efficacy of the treatment, e.g., by periodically testing the subject, e.g., evaluating one or more of the parameters or symptoms described above. Optionally, the methods can include quantifying improvement using parametric tests such as the GCS, FIM, or DRS described above, or using one or more brain imaging modalities, e.g., MR, fMR, CAT, or PET imaging, or using electrophysiologic measurements of conductivity or brain activity, e.g., Olfactory event-related potentials (OERPs) or Brainstem auditory evoked responses (BAERs).

### BACE1 Inhibitors

A number of BACE1 inhibitors are known in the art, including small molecules and inhibitory antibodies. BACE1 inhibitors include LY2886721 and LY2811376 (Lilly); MBI-1, MBI-3, MBI-5, and MK-8931 (Merck); E2609 (Eisai); RG7129 (Roche); TAK-070 (Takeda); CTS-21166 (CoMentis); AZD3293 and AZ4217 (AstraZeneca); HPP854 (High Point Pharmaceuticals); Ginsenoside Rg1 (CID 441923); Hispidin (CID310013); TDC (CID 5811533); Monacolin K (CID 53232); PF-05297909; SCH 1359113; Spirocyclic inhibitors (e.g., as described in Hunt et al., J Med Chem. 2013 Apr. 25; 56(8):3379-403, such as compound (R)-50); fluorine-substituted 1,3-oxazines (e.g., as described in Hilpert et al., J Med Chem. 2013 May 23; 56(10):3980-95, such as the CF3 substituted oxazine 89). Inhibitory antibodies include bispecific antibodies with one arm targeting BACE and the other recognizing transferrin receptor to boost brain penetrance (see, e.g., Yu et al., Sci Transl Med. 2011 May 25; 3(84):84ra44; Atwal et al., Sci Transl Med. 2011 May 25; 3(84):84ra43, and U.S. Pat. No. 8,772,457) and camelid antibodies that bind and inhibit BACE1 encoded by virus (see e.g., U.S. Pat. No. 8,568,717 and US20110091446).

These and other BACE1 inhibitors useful in the present methods are described in the following US Pre-Grant Publications: 20140286963; 20140275165; 20140235626; 20140228356; 20140228277; 20140186357; 20140179690; 20140112867; 20140057927; 20140051691; 20140011802; 20130289050; 20130217705; 20130210839; 20130108645; 20130105386; 20120258961; 20120245157; 20120245155; 20120245154; 20120238557; 20120237526; 20120232064; 20120214186; 20120202828; 20120202804; 20120190672; 20120172355; 20120171120; 20120148599; 20120094984; 20120093916; 20120064099; 20120015961; 20110288083; 20110237576; 20110207723; 20110158947; 20110152341; 20110152253; 20110091446; 20110071124; 20110033463; 20100317850; 20100285597; 20100273671; 20100221760; 20100144790; 20100132060; 20100093999; 20100075957; 20100063134; 20090258925; 20090209755; 20090176836; 20090162878; 20090136977; 20090081731; 20090060987; 20090042993; 20080124379; 20070224656; 20070185042; 20060216292; 20060182736; 20060178328; 20060052327; 20050196398; 20050048641; 20040248231; 20040220132; 20040162255; 20040132680; 20040063161; 20030194745; 20020159991; and 20020157122, and U.S. Pat. Nos. 8,772,457; 8,703,785; 8,568,717; 8,415,319; 8,288,354; 8,198,269; 8,183,219; 8,058,251; 7,829,694; 7,816,378; 7,618,948; 7,273,743; and 6,713,276.

### Inhibitory Oligonucleotides Targeting APLP2 or BACE1

As described above, the methods can include the administration of inhibitory oligonucleotides (“oligos”) targeting APLP2 and BACE1.

Sequences for human APLP2 are known in the art and include the following:

| | GenBank Acc. No. |
|---|---|
| Isoform | Nucleic acid |
| amyloid-like protein 2 isoform 2 precursor | NM_001142276.1 |
| amyloid-like protein 2 isoform 3 precursor | NM_001142277.1 |
| amyloid-like protein 2 isoform 4 precursor | NM_001142278.1 |
| amyloid-like protein 2 isoform 5 | NM_001243299.1 |
| amyloid-like protein 2 isoform 1 precursor | NM_001642.2 |

RNA sequences for APLP2 also include NR_024515.1 (variant 5) and NR_024516.1 (variant 6). Genomic sequence is at NG 029770.1, Range 5001 to 79991.

Sequences for human BACE1 are known in the art and include the following:

| | GenBank Acc. No. |
|---|---|
| Isoform | Nucleic acid |
| beta-secretase 1 isoform A preproprotein | NM_012104.4 |
| beta-secretase 1 isoform B preproprotein | NM_138972.3 |
| beta-secretase 1 isoform C preproprotein | NM_138971.3 |
| beta-secretase 1 isoform D preproprotein | NM_138973.3 |
| beta-secretase 1 isoform E | NM_001207048.1 |
| beta-secretase 1 isoform F | NM_001207049.1 |

Genomic sequence for human BACE1 is at NG 029372.1, range 5001 to 35571.

In some embodiments, the oligos hybridize to at least 7, 8, 9, 10, 11, 12, 13, 14, 15, 16, 17, 18, 19, 20, 21, 22, 23, 24, 25, 26, 27, 28, 29, 30 or more consecutive nucleotides of the target sequence.

In some embodiments, the methods include introducing into the cell an oligo that specifically binds, or is complementary, to BACE1 or APLP2. A nucleic acid that binds “specifically” binds primarily to the target, i.e., to BACE1 or APLP2 RNA but not to other non-target RNAs. The specificity of the nucleic acid interaction thus refers to its function (e.g., inhibiting BACE1 or APLP2) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. These methods can be used to treat a subject, e.g., a subject at risk for neurodegeneration following acute injury or with evidence of a chronic neurodegenerative disease, by administering to the subject a composition (e.g., as described herein) comprising an oligo that binds to BACE1 or APLP2. Examples of BACE1 or APLP2 target sequences are provided above.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease.

In some embodiments, the methods described herein include administering a composition, e.g., a sterile composition, comprising an oligo that is complementary to BACE1 or APLP2 sequence as described herein. Oligos for use in practicing the methods described herein can be an antisense or small interfering RNA, including but not limited to an shRNA or siRNA. In some embodiments, the oligo is a modified nucleic acid polymer (e.g., a locked nucleic acid (LNA) molecule), a gapmer, or a mixmer.

Oligos have been employed as therapeutic moieties in the treatment of disease states in animals, including humans. Oligos can be useful therapeutic modalities that can be configured to be useful in treatment regimens for the treatment of cells, tissues and animals, especially humans.

For therapeutics, an animal, preferably a human, suspected of having or being at risk of neurodegeneration is treated by administering an oligo in accordance with this disclosure. For example, in one non-limiting embodiment, the methods comprise the step of administering to the animal in need of treatment a therapeutically effective amount of an oligo as described herein.

### Oligonucleotides

Oligos useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), mixmers, gapmers, and other oligomeric compounds or oligonucleotide mimetics that hybridize to at least a portion of BACE1 or APLP2 and modulate its function. In some embodiments, the oligos include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO2010/040112.

In some embodiments, the oligos are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such oligos; for example, an oligo 30 nucleotides in length may have a portion of 15 bases that is complementary to the targeted BACE1 or APLP2 RNA. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies oligos having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the oligo comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligos are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligos of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligo comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO 2008/043753 and WO2007031091 and include compounds of the following formula.

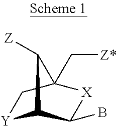

where X and Y are independently selected among the groups —O—, —S—, —N(H)—, N(R)—, —CH2- or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligomer of the invention comprises at least one LNA unit according any of the formulas

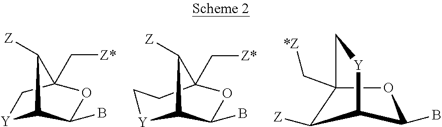

wherein Y is —O—, —S—, —NH—, or N(R^{H}); Z and Z* are independently selected among an internucleoside linkage, a terminal group or a protecting group; B constitutes a natural or non-natural nucleotide base moiety, and R^{H} is selected from hydrogen and C₁₋₄-alkyl. Preferably, the Locked Nucleic Acid (LNA) used in an oligomeric compound, such as an antisense oligonucleotide, as described herein comprises at least one nucleotide comprises a Locked Nucleic Acid (LNA) unit according any of the formulas shown in Scheme 2 of PCT/DK2006/000512 (WO2007031091).

Preferably, the LNA used in the oligomer of the invention comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, -0-PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown in scheme 3:

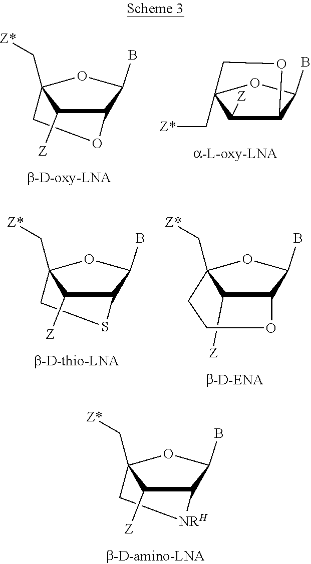

The term “thio-LNA” comprises a locked nucleotide in which at least one of X or Y in the general formula above is selected from S or —CH2-S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail below. One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligos can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2<0>C. (Sanghvi, in Crooke, and Lebleu, eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligos can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5- bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3- deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5- propynylcytosine. 5-methylcytosine substitutions have been shown to increase nucleic acid duplex stability by 0.6-1.2° C. (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligos are chemically linked to one or more moieties or conjugates that enhance the activity, cellular distribution, or cellular uptake of the oligonucleotide. For example, one or more oligos, of the same or different types, can be conjugated to each other; or oligos can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl- rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac- glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The oligos useful in the present methods are sufficiently complementary to the target BACE1 or APLP2, e.g., hybridize sufficiently well and with sufficient biological functional specificity, to give the desired effect. “Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of an oligo is capable of hydrogen bonding with a base at the corresponding position of BACE1 or APLP2, then the bases are considered to be complementary to each other at that position. 100% complementarity is not required. As noted above, oligos can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

Routine methods can be used to design an oligo that binds to a selected site sequence with sufficient specificity. In some embodiments, the methods include using bioinformatics methods known in the art to identify regions of secondary structure, e.g., one, two, or more stem-loop structures, or pseudoknots, and selecting those regions to target with an oligo. For example, methods of designing oligonucleotides similar to the oligos described herein, and various options for modified chemistries or formats, are exemplified in Lennox and Behlke, Gene Therapy (2011) 18: 1111-1120.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. One having skill in the art armed with the sequences provided herein will be able, without undue experimentation, to identify further preferred regions to target with complementary oligos.

In the context of the present disclosure, hybridization means base stacking and hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as the term is used in the art, refers to the capacity for precise pairing between two nucleotides. For example, if a nucleotide at a certain position of an oligonucleotide is capable of hydrogen bonding with a nucleotide at the same position of BACE1 or APLP2 molecule, then the oligo and the BACE1 or APLP2 molecule are considered to be complementary to each other at that position. The oligos and the BACE1 or APLP2 molecule are complementary to each other when a sufficient number of corresponding positions in each molecule are occupied by nucleotides that can hydrogen bond with each other through their bases. Thus, “specifically hybridizable” and “complementary” are terms which are used to indicate a sufficient degree of complementarity or precise pairing such that stable and specific binding occurs between the oligo and the BACE1 or APLP2 molecule. For example, if a base at one position of an oligo is capable of hydrogen bonding with a base at the corresponding position of BACE1 or APLP2, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridizable when binding of the sequence to the target BACE1 or APLP2 molecule interferes with the normal function of BACE1 or APLP2 to cause a loss of activity or expression and there is a sufficient degree of complementarity to avoid non-specific binding of the sequence to non-target sequences under conditions in which avoidance of the non-specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the oligos useful in the methods described herein have at least 80% sequence complementarity to a target region within the target nucleic acid, e.g., 90%, 95%, or 100% sequence complementarity to the target region within BACE1 or APLP2. For example, an antisense compound in which 18 of 20 nucleobases of the antisense oligonucleotide are complementary, and would therefore specifically hybridize, to a target region would represent 90 percent complementarity. Percent complementarity of an oligo with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the invention that hybridize to BACE1 or APLP2 are identified through routine experimentation. In general the oligos must retain specificity for their target, i.e., either do not directly bind to, or do not directly significantly affect expression levels of, transcripts other than the intended target.

Target-specific effects, with corresponding target-specific functional biological effects, are possible even when the oligo exhibits non-specific binding to a large number of non-target RNAs. For example, short 8 base long oligos that are fully complementary to BACE1 or APLP2 may have multiple 100% matches to hundreds of sequences in the genome, yet may produce target-specific effects, e.g. upregulation of a specific target gene through inhibition of BACE1 or APLP2 activity. 8-base oligos have been reported to prevent exon skipping with with a high degree of specificity and reduced off-target effect. See Singh et al., RNA Biol., 2009; 6(3): 341-350. 8-base oligos have been reported to interfere with miRNA activity without significant off-target effects. See Obad et al., Nature Genetics, 2011; 43: 371-378.

For further disclosure regarding oligos, please see US2010/0317718 (antisense oligos); US2010/0249052 (double-stranded ribonucleic acid (dsRNA)); US2009/0181914 and US2010/0234451 (LNA molecules); US2007/0191294 (siRNA analogues); US2008/0249039 (modified siRNA); and WO2010/129746 and WO2010/040112 (oligos).

### Antisense

In some embodiments, the oligos are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to BACE1 or APLP2 in vitro, and are expected to inhibit the activity of BACE1 or APLP2 in vivo. Thus, oligonucleotides are chosen that are sufficiently complementary to the target, i.e., that hybridize sufficiently well and with sufficient biological functional specificity, to give the desired effect.

### Modified Bases, Including Locked Nucleic Acids (LNAs)

In some embodiments, the oligos used in the methods described herein comprise one or more modified bonds or bases. Modified bases include phosphorothioate, methylphosphonate, peptide nucleic acids, or locked nucleic acids (LNAs). Oligos that have been modified (locked nucleic acid—LNA) have demonstrated the “on target” specificity of this approach. Preferably, the modified nucleotides are part of locked nucleic acid molecules, including [alpha]-L-LNAs. LNAs include ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., BACE1 or APLP2 sequences as described herein.

The modified base/LNA molecules can include molecules comprising, e.g., 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the BACE1 or APLP2. The modified base/LNA molecules can be chemically synthesized using methods known in the art.

The modified base/LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of a modified base/LNA molecule; for example, a series of oligonucleotides of 10-30 nucleotides spanning the length of a target BACE1 or APLP2 can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing modified base/LNA molecules are known in the art; for example, LNA sequences will bind very tightly to other LNA sequences, so it is preferable to avoid significant complementarity within an LNA molecule. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNA molecules see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

As demonstrated herein, LNA molecules can be used as a valuable tool to manipulate and aid analysis of BACE1 or APLP2 RNAs. Advantages offered by an LNA molecule-based system are the relatively low costs, easy delivery, and rapid action. While other oligos may exhibit effects after longer periods of time, LNA molecules exhibit effects that are more rapid, e.g., a comparatively early onset of activity, are fully reversible after a recovery period following the synthesis of new BACE1 or APLP2 molecules, and occur without causing substantial or substantially complete RNA cleavage or degradation. One or more of these design properties may be desired properties of the oligos of the invention. Additionally, LNA molecules make possible the systematic targeting of domains within much longer nuclear transcripts. The LNA technology enables high-throughput screens for functional analysis of BACE1 or APLP2 RNAs and also provides a novel tool to manipulate chromatin states in vivo for therapeutic applications.

In various related aspects, the methods described herein include using LNA molecules to target BACE1 or APLP2 for a number of uses, including as a research tool to probe the function of BACE1 or APLP2, e.g., in vitro or in vivo. The methods include selecting one or more desired BACE1 or APLP2 sequences, designing one or more LNA molecules that target the BACE1 or APLP2 sequences, providing the designed LNA molecule, and administering the LNA molecule to a cell or animal.

In still other related aspects, the LNA molecules targeting BACE1 or APLP2 as described herein can be used to create animal or cell models of conditions associated with altered BACE1 or APLP2 expression.

### Antagomirs

In some embodiments, the oligo is an antagomir. Antagomirs are chemically modified antisense oligonucleotides that can target BACE1 or APLP2. For example, an antagomir for use in the methods described herein can include a nucleotide sequence sufficiently complementary to hybridize to BACE1 or APLP2 target sequence of about 12 to 25 nucleotides, preferably about 15 to 23 nucleotides.

In some embodiments, antagomirs include a cholesterol moiety, e.g., at the 3′-end. In some embodiments, antagomirs have various modifications for RNase protection and pharmacologic properties such as enhanced tissue and cellular uptake. For example, in addition to the modifications discussed above for antisense oligos, an antagomir can have one or more of complete or partial 2′-O-methylation of sugar and/or a phosphorothioate backbone. Phosphorothioate modifications provide protection against RNase or other nuclease activity and their lipophilicity contributes to enhanced tissue uptake. In some embodiments, the antagomir cam include six phosphorothioate backbone modifications; two phosphorothioates are located at the 5′-end and four at the 3′-end, but other patterns of phosphorothioate modification are also commonly employed and effective. See, e.g., Krutzfeldt et al., Nature 438, 685-689 (2005); Czech, N Engl J Med 2006; 354:1194-1195 (2006); Robertson et al., Silence. 1:10 (2010); Marquez and McCaffrey, Hum Gene Ther. 19(1):27-38 (2008); van Rooij et al., Circ Res. 103(9):919-928 (2008); and Liu et al., Int. J. Mol. Sci. 9:978-999 (2008).

In general, the design of an antagomir avoids target RNA degradation due to the modified sugars present in the molecule. The presence of an unbroken string of unmodified sugars supports RNAseH recruitment and enzymatic activity. Thus, typically the design of an antagomir will include bases that contain modified sugar (e.g., LNA), at the ends or interspersed with natural ribose or deoxyribose nucleobases.

Antagomirs useful in the present methods can also be modified with respect to their length or otherwise the number of nucleotides making up the antagomir. In some embodiments, the antagomirs must retain specificity for their target, i.e., must not directly bind to, or directly significantly affect expression levels of, transcripts other than the intended target. In some embodiments, antagomirs may exhibit nonspecific binding that does not produce significant undesired biologic effect, e.g., the antagomirs do not affect expression levels of non-target transcripts or their association with regulatory proteins or regulatory RNAs.

Interfering RNA, Including siRNA/shRNA

In some embodiments, the oligo sequence that is complementary to BACE1 or APLP2 can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

### Ribozymes

In some embodiments, the oligos are ribozymes. Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Man, 1995 J. Med. Chem. 38, 2023-2037). Enzymatic nucleic acid molecules can be designed to cleave specific caRNA targets within the background of cellular RNA. Such a cleavage event renders the caRNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 MM) concentrations of Mg cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Making and Using Oligos

The nucleic acid sequences used to practice the methods described herein, whether

RNA, cDNA, genomic DNA, vectors, viruses or hybrids thereof, can be isolated from a variety of sources, genetically engineered, amplified, and/or expressed/generated recombinantly. If desired, nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)).

Preferably, oligos of the invention are synthesized chemically. Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066; WO/2008/043753 and WO/2008/049085, and the refences cited therein.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl(2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

It is understood that any of the modified chemistries or formats of oligos described herein can be combined with each other, and that one, two, three, four, five, or more different types of modifications can be included within the same molecule.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Modification Patterns

In some embodiments, the inhibitory oligonucleotide comprises locked nucleic acids (LNA), ENA modified nucleotides, 2′-O-methyl nucleotides, or 2′-fluoro-deoxyribonucleotides. In some embodiments, the inhibitory oligonucleotide comprises alternating deoxyribonucleotides and 2′-fluoro-deoxyribonucleotides. In some embodiments, the inhibitory oligonucleotide comprises alternating deoxyribonucleotides and 2′-O-methyl nucleotides. In some embodiments, the inhibitory oligonucleotide comprises alternating deoxyribonucleotides and ENA modified nucleotides. In some embodiments, the inhibitory oligonucleotide comprises alternating deoxyribonucleotides and locked nucleic acid nucleotides. In some embodiments, the inhibitory oligonucleotide comprises alternating locked nucleic acid nucleotides and 2′-O-methyl nucleotides.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the inhibitory oligonucleotide comprises phosphorothioate internucleotide linkages. In some embodiments, the single stranded oligonucleotide comprises phosphorothioate internucleotide linkages between at least two nucleotides. In some embodiments, the single stranded oligonucleotide comprises phosphorothioate internucleotide linkages between all nucleotides.

It should be appreciated that the oligonucleotide can have any combination of modifications as described herein.

As an example, the oligonucleotide may comprise a nucleotide sequence having one or more of the following modification patterns.

### (a) (X)Xxxxxx, (X)xXxxxx, (X)xxXxxx, (X)xxxXxx, (X)xxxxXx and (X)xxxxxX,

### (b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

### (c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

### (d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

### (e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270: 1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 372( ):137-41).

### Additional Sequence Structural Information

The inhibitory oligonucleotides described herein may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The inhibitory oligonucleotides have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than the gene of interest. The oligonucleotide is expected to have a reduced likelihood of having off-target effects. The threshold level of sequence identity may be 50%, 60%, 70%, 80%, 85%, 90%, 95%, 99% or 100% sequence identity.

The inhibitory oligonucleotides may have a sequence that is complementary to a region that encodes an RNA that forms a secondary structure comprising at least two single stranded loops. In some embodiments, oligonucleotides that are complementary to a region that encodes an RNA that forms a secondary structure comprising one or more single stranded loops (e.g., at least two single stranded loops) have a greater likelihood of being active (e.g., of being capable of activating or enhancing expression of a target gene) than a randomly selected oligonucleotide. In some cases, the secondary structure may comprise a double stranded stem between the at least two single stranded loops. Accordingly, the area of complementarity between the oligonucleotide and the nucleic acid region may be at a location of the PRC2 associated region that encodes at least a portion of at least one of the loops. In some embodiments, the predicted secondary structure RNA (e.g., of the BACE1 or APLP2 sequence) containing the nucleic acid region is determined using RNA secondary structure prediction algorithms, e.g., RNAfold, mfold. In some embodiments, oligonucleotides are designed to target a region of the RNA that forms a secondary structure comprising one or more single stranded loop (e.g., at least two single stranded loops) structures which may comprise a double stranded stem between the at least two single stranded loops.

The inhibitory oligonucleotide may have a sequence that is has greater than 30% G-C content, greater than 40% G-C content, greater than 50% G-C content, greater than 60% G-C content, greater than 70% G-C content, or greater than 80% G-C content. The inhibitory oligonucleotide may have a sequence that has up to 100% G-C content, up to 95% G-C content, up to 90% G-C content, or up to 80% G-C content.

In some embodiments, the region of complementarity of the inhibitory oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of BACE1 or APLP2 as known in the art or disclosed herein. In some embodiments, the region of complementarity is complementary with at least 8, 10, 12, 14, 16, 18, or 20 consecutive nucleotides of BACE1 or APLP2 as known in the art or disclosed herein.

### Pharmaceutical Compositions and Methods of Administration

The methods described herein can include the administration of pharmaceutical compositions and formulations comprising BACE1 inhibitors and/or oligonucleotides designed to target BACE1 or APLP2.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The oligos can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an oligo can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation.

In some embodiments, the methods described herein can include co-administration with other drugs or pharmaceuticals, e.g., compositions for providing cholesterol homeostasis. For example, the oligos can be co-administered with drugs for treating or reducing risk of a disorder described herein.

### Dosage

An “effective amount” is an amount sufficient to effect beneficial or desired therapeutic or prophylactic results, e.g., to treat, ameliorate, delay, or reduce the severity, progression, risk, or onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered, e.g., from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1: A Reliable, Reproducible Paradigm of Acute Axonal Injury, Neurodegeneration, and Neuroregeneration

This example describes a reliable, reproducible paradigm of surgical injury to axons of OSNs after the pass through the cribiform plate into the brain, but prior to innervating the olfactory bulb and establishing synapses, modeling the shear injury and diffuse axonal injury observed in humans (12).

Preoperative Procedure:

The animals were anesthetized by inhalation, using 2% isoflurane, supplemented with equal amounts of oxygen, or by ketamine and xylazine.

Surgery/Procedure:

a. After anesthesia as described above, scalp was shaved clean of fur and swabbed with povinde/iodine solution. Bupivocaine 0.25% was injected subcutaneously at the site of the incision. A longitudinal incision was made and a circular section of scalp cut between eyes to between ears (approx. 10 mm diameter). Mouse was placed in a stereotaxic apparatus.

b. Connective tissue and aponeurosis were cleaned with a swab.

c. The following cranial windowing procedure was modified from Yuan et al (Cancer Research (1994). 54, 4564-68) and from Spires-Jones et al. (Methods, 53(3): 201-207, 2011). Using a high speed microdrill with a 0.4 mm burr tip, a 1 mm diameter hole was made. Cold, sterile PBS was applied during the process to avoid thermal injury to the cortex.

d. A wire loop was slid along the anterior surface of the cribiform plate on the right side only to a depth of 1.5 mm to sever about 20% of axons of olfactory sensory neurons.

e. The animal was removed from the microscope and placed on a heating pad to recover, with temperature monitoring while the mouse woke up. Buprenorphine (mouse: 0.05-0.1 mg/kg) was administrated immediately following olfactory axotomy.

Post surgical/procedure care:

Animals were monitored 2 times per day (such as early morning and late afternoon) for 3 days. Buprenorphine (mouse: 0.05-0.1 mg/kg) was be administrated every 12 h following olfactory axotomy for 72 hours.

The mice were sacrificed 3 days, 7 days or 14 days after the procedure by Avertin IP injection, intracardiac perfusion with PBS, and dissection of the olfactory epithelium and olfactory bulb. This tissue was analyzed by immunohistochemistry to determine the loss of olfactory neurons.

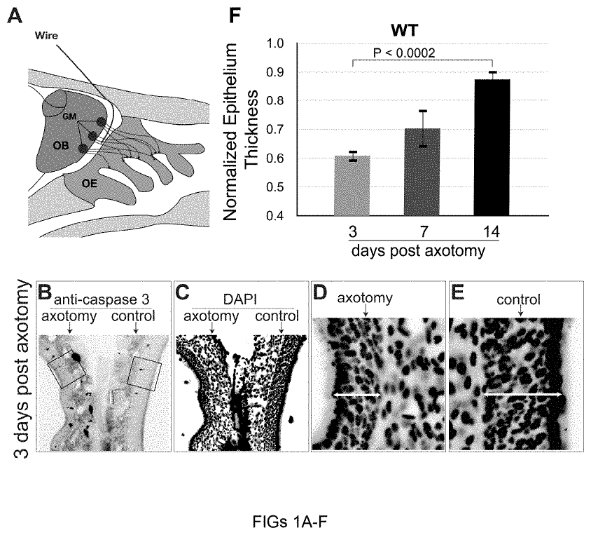

The epithelium of the right dorso-medial and septal regions contains neurons with injured axons, demonstrated by induction of cleaved caspase 3 in the axon bundles of these neurons 3 days post-axotomy. By 24 hours post-axotomy, there was induction of cleaved caspase 3 in both the soma of olfactory sensory neurons (OSNs) and the axon bundles of the injured side. Unilateral OSN axotomy resulted in robust axonal cleaved caspase 3 activation and retrograde propagation of activated caspase 3 and cell death of the majority of mature OSNs in affected regions at 3 days at injury, followed by regeneration and nearly full recovery by 14 days post injury (FIG. 1).

### Example 2: No Neuroprotection Offered by Loss of BACE1 or APP

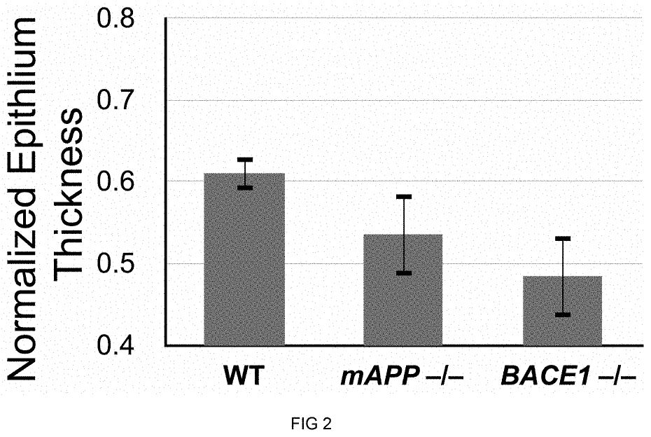

This massive OSN degeneration and epithelial thinning observed 3 days after acute axonal injury was present in BACE1−/− mice. Similarly, the same degree of neuronal loss was seen in APP−/− mice (FIG. 2). The data indicate that the production of A3 is not an essential mechanism of neurodegeneration of OSNs after acute axonal injury. Consistent with this interpretation is the finding that axotomy of OSNs did not lead to worse neurodegeneration in a mouse line that overexpressed wild type human APP in most OSNs, and produced human Aβ peptide.

APLP2 shares high sequence similarity and functional architecture with the amyloid precursor protein (APP), and is enriched in many types of cells in mammalian neural structures (21). However, little is known about the physiological function of APLP2. The APLP2 gene is highly expressed (#27 overall) in mouse OSNs as revealed by RNA-seq of purified OSNs isolated by fluorescence activated cell sorting (22). Mice deficient of both APP and APLP2 suffer postnatal lethality (23). However, mice deficient of APP or APLP2 are viable with only minor reported phenotypes (24). Although no loss in neuronal function has been reported in APLP2 knockout (−/−) mice (25), RNA microarray data derived from cortical neurons in vivo revealed increased expression of clusters of genes involved in neurogenesis and the negative regulation of cell apoptosis (26). Intriguingly, several studies revealed that cellular APLP2 gene expression is increased in response to stresses in vitro and in vivo, including trophic factor withdrawal-induced death of neuronal PC12 cells (27), olfactory bulbectomy (28) and lesioning of the nigrostriatal pathway (29). Moreover, the APLP2 protein is markedly upregulated in human brains following TBI.

The present inventors discovered that the amyloid like precursor protein 2 (APLP2) and the beta site-APP cleaving enzyme 1 (BACE1) plays a critical role in the physiological programmed cell death of olfactory sensory neurons (OSNs) in adult mice in vivo:

### Example 3. Decreased Apoptosis of Olfactory Sensory Neurons in APLP2−/− Mice

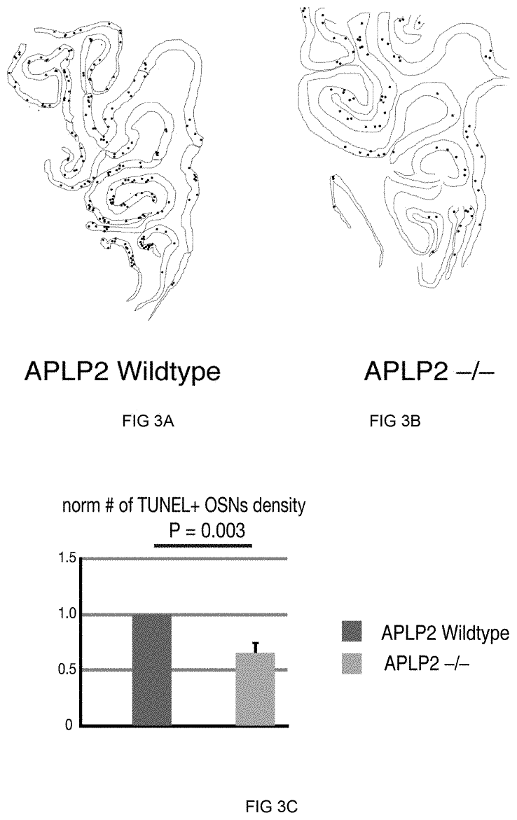

Terminal deoxynucleotidyl transferase dUTP nick end labeling (TUNEL) assay was applied to identify OSNs undergoing apoptosis. TUNEL staining of the olfactory epithelium revealed a 30% reduction of the density of TUNEL-positive OSNs in APLP2−/− mice relative to controls (FIG. 3).

### Example 4. Decreased OSN Neurogenesis and Increased OSN Half-Life in APLP2−/− Mice

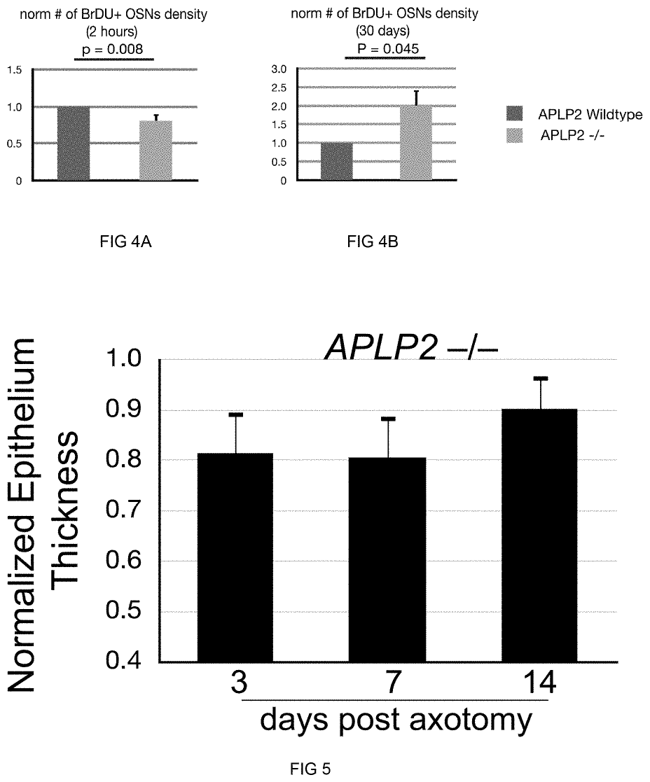

The density of bromo-deoxyuridine (BrDU)-labelled OSNs in olfactory epithelial was quantified at two different time points (2 hours and 30 days) after BrDu injection. In APLP2−/− mice, there is 20% of reduction of the density at 2 hours, indicating a significant reduction in the rate of neurogenesis. Moreover, we demonstrated an increased half life of OSNs by quantifying BrDU+ OSNS at 30 days post-injection (FIG. 4). Together, these results indicate that APLP2 is essential for the physiological programmed cell death of olfactory neurons.

### Example 5. OSN Deafferentation Induced Neuronal Degeneration is Rescued in APLP2−/− Mice

In APLP2−/− mice, acute unilateral deafferentation of OSNs by an axotomy results in markedly reduced OSN loss, preserving epithelial thickness that is only 20% thinner to the untreated side. These results indicate that the deficiency of APLP2 protects OSNs from degeneration in response to acute injury without impairing recovery of epithelial thickness after 14 days (FIG. 5).

By contrast, APP is not necessary for physiological apoptosis and deletion of APP does not protect OSNs from profound death following axotomy in our TBI paradigm (FIG. 2). APLP2 is a type I transmembrane protein that can function as a receptor and as a ligand (or both).

### Example 6. OSN Deafferentation Induced Neuronal Degeneration is Rescued in BACE1+/− Partial Knockout Mice, but the Neuronal Degeneration is Exacerbated in BACE1−/− Mice

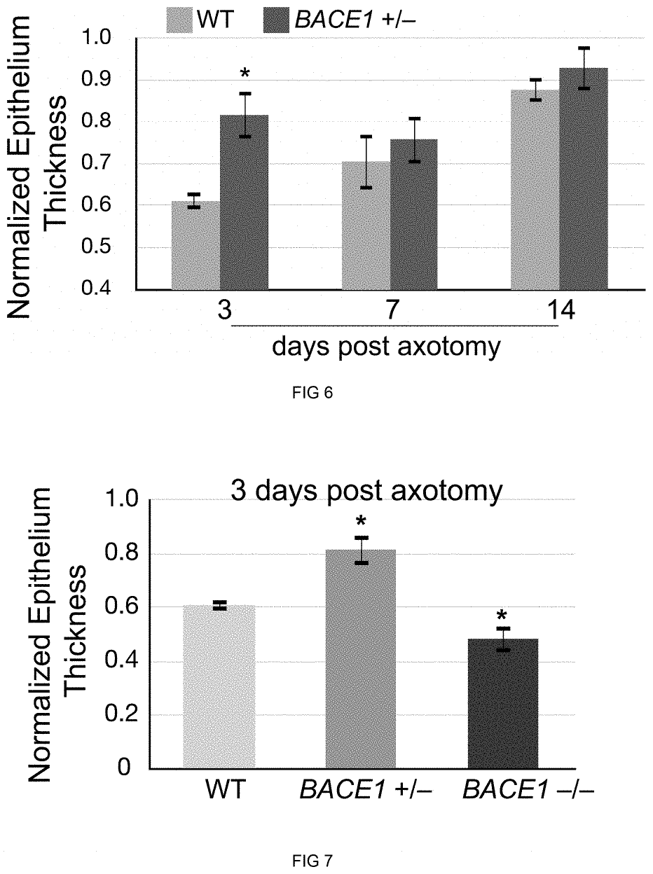

We also found that adult mice genetically modified to disrupt expression of the BACE1 protease from one allele (heterozygous knockout) also demonstrated protection of olfactory sensory neurons three days following axonal shearing. In BACE1+/− mice, OSN loss is markedly reduced after acute OSN axotomy (FIG. 6), preserving epithelial thickness that is comparable to effect observed in the APLP2 knockout mouse (FIG. 5). On the other hand, the degree of epithelial loss in BACE1−/− mice is exacerbated (FIG. 7). These results indicate that the partial deficiency of BACE] protects OSNs from degeneration in response to acute axonal injury, which is a realistic goal for pharmacologic therapy. Moreover, the complete loss of BACE1 function resulting in a possible exacerbation of neuronal loss suggests that the dose-response curve for BACE1 inhibitors may be U-shaped—indicating that careful titration of BACE1 inhibitors will be required to maximize the therapeutic response of BACE1 inhibitors. Since BACE1 has received much attention as a pharmacologic target for AD, many existing well characterized pharmacologic inhibitors of BACE1's protease function are available to afford a partial loss of BACE1's function.

### Example 7: Acute Inhibition of BACE by Inhibitors Confers a Neuroprotective Response Following Acute Axotomy Injury

To determine whether acute inhibition of BACE1 in adult mice is sufficient to provide protection of OSNs following axonal injury, inhibitors of BACE1 with excellent brain penetration are administered. For example, MBI-1 is an orally available inhibitor (Kᵢ BACE1 1.7 nM; cell ED₅₀=11 nM; rat cortex ED₅₀=6 mg/kg/day) that can achieve 80-90% inhibition of BACE1 in the brain as measured by production of human Aβ peptide. Since data indicates a non-linear response of genetic reduction of BACE1 (haploinsufficiency confers neuroprotection, whereas the BACE1 null mice have exacerbated neuronal loss), a dosing study is conducted. Based on previous studies in mice, MBI-1 is administered by oral gavage at the following doses (vehicle (20% hydroxypropyl-β-cyclodextrin; 0% reduction of BACE1 activity); 3 mg/kg/d (25% reduction of BACE1 activity); 10 mg/kg/d (50% reduction of BACE1 activity); 40 mg/kg/d (75% reduction of BACE1 activity). Alternatively, MBI-3 (Kᵢ BACE1 0.9 nM; HEK-293 IC50=1.0 nM; Asp protease >10000) can be administered in diet (Tg V diet by Research Diets, Inc. (Merck) at 3 mg/kg/day 25% inhibition; 10 mg/kg/day 50% inhibition; and 40 mg/kg/day 75% inhibition) to achieve similar levels of inhibition. MBI-1 is administered twice a day by oral gavage, and MBI-3 is administered by continuous access to the diet for 4 d prior to the axonal injury paradigm and continued until the animals are sacrificed 3 d later. At 3 d post injury, the mice are analyzed for loss of OSNs using either epithelial thickness (as shown in the preliminary data) or quantitation using flow cytometry. Inhibition of BACE1 in OSNs in vivo is confirmed by performing a Western blot on solubilized membrane extracts from OSNs following treatment with MBI-1. APP and APLP2 are two validated substrates of BACE1 which are highly expressed in OSNs. Steady state levels of the C99 products of mouse APP and APLP2 are compared using C-terminal specific antibodies (6300 (Sigma) and CT12 for APLP2 (G. Thinakaran, Univ. of Chicago)). Extracts from BACE1+/− and BACE1−/− mice will serve as positive controls for this Western blot.

To determine the degree of loss and recovery of OSNs in BACE1+/− mice following acute axotomy before and after the administration of a potent BACE inhibitor, the experiment described above is repeated in BACE1+/− knockout mice to demonstrate specificity of the pharmacological agent. Any neuroprotective effects conferred by MBI-1 and/or MBI-3 are expected to be absent in the BACE1+/− mouse. It is possible that there will be no evidence of neuroprotection since no neuroprotection was observed in the BACE1−/− mouse. Based on the data, it is anticipated that 50% reduction of BACE1 activity will confer a protective response if acute inhibition is sufficient to confer neuroprotection. Administration of MBI-1 immediately after the axonal injury is expected to be sufficient to mediate protection of OSNs.

### REFERENCES

1. Langlois J A, Rutland-Brown W, Wald M M. The epidemiology and impact of traumatic brain injury: a brief overview. The Journal of head trauma rehabilitation. 2006; 21(5):375-8.
2. Menon D K, Schwab K, Wright D W, Maas A I, Demographics, Clinical Assessment Working Group of the I, et al. Position statement: definition of traumatic brain injury. Archives of physical medicine and rehabilitation. 2010; 91(11): 1637-40.
3. Xiong Y, Mahmood A, Chopp M. Animal models of traumatic brain injury. Nat Rev Neurosci. 2013; 14(2):128-42
4. Masel B E, DeWitt D S. Traumatic brain injury: a disease process, not an event. Journal of neurotrauma. 2010; 27(8):1529-40
5. Raghupathi R. Cell death mechanisms following traumatic brain injury. Brain Pathol. 2004; 14(2):215-22
6. Buki A, Okonkwo D O, Wang K K, Povlishock J T. Cytochrome c release and caspase activation in traumatic axonal injury. J Neurosci. 2000; 20(8):2825-34.
7. Yokobori S, Mazzeo A T, Hosein K, Gajavelli S, Dietrich W D, Bullock M R. Preconditioning for traumatic brain injury. Translational stroke research. 2013; 4(1):25-39
8. Marklund N, Bakshi A, Castelbuono D J, Conte V, McIntosh T K. Evaluation of pharmacological treatment strategies in traumatic brain injury. Current pharmaceutical design. 2006; 12(13):1645-80.
9. Drummond M, Douglas J, Olver J. ‘If I haven't got any smell . . . I'm out of work’: consequences of olfactory impairment following traumatic brain injury. Brain injury: [BI]. 2013; 27(3):332-45
10. Haxel B R, Grant L, Mackay-Sim A. Olfactory dysfunction after head injury. The Journal of head trauma rehabilitation. 2008; 23(6):407-13
11. Fortin A, Lefebvre M B, Ptito M. Traumatic brain injury and olfactory deficits: the tale of two smell tests! Brain injury: [BI]. 2010; 24(1):27-33
12. Schofield P W, Moore T M, Gardner A. Traumatic Brain Injury and Olfaction: A Systematic Review. Frontiers in neurology. 2014; 5:5.
13. Costanzo R M, Miwa T. Posttraumatic olfactory loss. Advances in oto-rhino-laryngology. 2006; 63:99-107
14. Gudziol V, Wolff-Stephan S, Aschenbrenner K, Joraschky P, Hummel T. Depression resulting from olfactory dysfunction is associated with reduced sexual appetite—a cross-sectional cohort study. The journal of sexual medicine. 2009; 6(7): 1924-9.
15. Sumner D. Post-Traumatic Anosmia. Brain. 1964; 87:107-20.
16. Henkin R I, Velicu I, Schmidt L. An open-label controlled trial of theophylline for treatment of patients with hyposmia. The American journal of the medical sciences. 2009; 337(6):396-406.
17. Cao L, Schrank B R, Rodriguez S, Benz E G, Moulia T W, Rickenbacher G T, et al. Abeta alters the connectivity of olfactory neurons in the absence of amyloid plaques in vivo. Nat Commun. 2012; 3:1009.
18. Schwob J E. Neural regeneration and the peripheral olfactory system. The Anatomical record. 2002; 269(1):33-49.
19. Strich S J. Diffuse degeneration of the cerebral white matter in severe dementia following head injury. J Neurol Neurosurg Psychiatry. 1956; 19(3):163-85.
20. Marklund N, Farrokhnia N, Hanell A, Vanmechelen E, Enblad P, Zetterberg H, et al. Monitoring of beta-Amyloid Dynamics after Human Traumatic Brain Injury. Journal of neurotrauma. 2014; 31(1):42-55.
21. Walsh D M, Selkoe D J. A beta oligomers—a decade of discovery. J Neurochem. 2007; 101(5): 1172-84.
22. Lyons D B, Allen W E, Goh T, Tsai L, Bamea G, Lomvardas S. An epigenetic trap stabilizes singular olfactory receptor expression. Cell. 2013; 154(2):325-36.
23. Heber S, Herms J, Gajic V, Hainfellner J, Aguzzi A, Rulicke T, et al. Mice with combined gene knock-outs reveal essential and partially redundant functions of amyloid precursor protein family members. J Neurosci. 2000; 20(21):7951-63.
24. von Koch C S, Zheng H, Chen H, Trumbauer M, Thinakaran G, van der Ploeg L H, et al. Generation of APLP2 K O mice and early postnatal lethality in APLP2/APP double K O mice. Neurobiol Aging. 1997; 18(6):661-9.
25. Weyer S W, Klevanski M, Delekate A, Voikar V, Aydin D, Hick M, et al. APP and APLP2 are essential at PNS and CNS synapses for transmission, spatial learning and LTP. EMBO J. 2011; 30(11):2266-80. Epub 2011/04/28
26. Aydin D, Filippov M A, Tschape J A, Gretz N, Prinz M, Eils R, et al. Comparative transcriptome profiling of amyloid precursor protein family members in the adult cortex. BMC Genomics. 2011; 12:160.
27. Araki W, Wurtman R J. Increased expression of amyloid precursor protein and amyloid precursor-like protein 2 during trophic factor withdrawal-induced death of neuronal PC12 cells. Brain Res Mol Brain Res. 1998; 56(1-2):169-77.
28. Cowan C M, Thai J, Krajewski S, Reed J C, Nicholson D W, Kaufmann S H, et al. Caspases 3 and 9 send a pro-apoptotic signal from synapse to cell body in olfactory receptor neurons. J Neurosci. 2001; 21(18):7099-109.
29. Shen Y, Yu Y, Guo H, Tang Z, Yu F S, Zhou J. Identification and comparative analysis of differentially expressed proteins in rat striatum following 6-hydroxydopamine lesions of the nigrostriatal pathway: up-regulation of amyloid precursor-like protein 2 expression. Eur J Neurosci. 2002; 16(5):896-906.
30. Cao L, Rickenbacher G T, Rodriguez S, Moulia T W, Albers M W. The precision of axon targeting of mouse olfactory sensory neurons requires the BACE1 protease. Sci Rep. 2012; 2(231):1-8..
31. Hitt B, Riordan S M, Kukreja L, Eimer W A, Rajapaksha T W, Vassar R. beta-Site amyloid precursor protein (APP)-cleaving enzyme 1 (BACE1)-deficient mice exhibit a close homolog of L1 (CHL1) loss-of-function phenotype involving axon guidance defects. The Journal of biological chemistry. 2012; 287(46):38408-25.
32. Tsai L, Barnea G. A critical period defined by axon-targeting mechanisms in the murine olfactory bulb. Science. 2014; 344(6180):197-200.
33. Ma L, Wu Y, Qiu Q, Scheerer H, Moran A, Yu C R. A developmental switch of axon targeting in the continuously regenerating mouse olfactory system. Science. 2014; 344(6180):194-7.
34. Yu D, Pendergraff H, Liu J, Kordasiewicz H B, Cleveland D W, Swayze E E, et al. Single-stranded RNAs use RNAi to potently and allele-selectively inhibit mutant huntingtin expression. Cell. 2012; 150(5):895-908.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject after acute trauma to the head, viral encephalitis, or other causes of acute neurodegeneration, and/or after acute vascular insults, including ischemic and hemorrhagic strokes, the method comprising administering a therapeutically effective amount of one or more of:
an inhibitor of BACE1;
an inhibitory oligonucleotide targeting BACE1; or
an inhibitory oligonucleotide targeting APLP2.

2. A method of treating a subject who is at high risk for head trauma, the method comprising administering a prophylactically effective amount of one or more of:
an inhibitor of BACE1;
an inhibitory oligonucleotide targeting BACE1; or
an inhibitory oligonucleotide targeting APLP2.

3. The method of claim 2, wherein the subject is an athlete or soldier.

4. A method of treating a chronic neurodegenerative disease in a subject, the method comprising administering a therapeutically effective amount of an inhibitory oligonucleotide targeting APLP2.

5. The method of claim 4, wherein the chronic neurodegenerative disease is Alzheimer's disease, Parkinson's disease, Huntington's disease, or frontotemporal dementia.

6. The method of claims 1-5, wherein the treatment promotes survival of neurons in the central nervous system.

7. The method of claim 6, wherein the treatment promotes survival of olfactory sensory neurons.

8. The method of claims 1-3, wherein the inhibitor of BACE1 is a small molecule or antibody that binds to BACE1.

9. The method of claim 8, wherein the inhibitor of BACE1 is selected from the group consisting of LY2886721 and LY2811376 (Lilly); MBI-1, MBI-3, MBI-5, and MK-8931 (Merck); E2609 (Eisai); RG7129 (Roche); TAK-070 (Takeda); CTS-21166 (CoMentis); AZD3293 and AZ4217 (AstraZeneca); HPP854 (High Point Pharmaceuticals); Ginsenoside Rg1 (CID 441923); Hispidin (CID310013); TDC (CID 5811533); Monacolin K (CID 53232); PF-05297909; SCH 1359113; Spirocyclic inhibitors (e.g., compound (R)-50); fluorine-substituted 1,3-oxazines (e.g., the CF3 substituted oxazine 89).

10. The method of claim 8, wherein the inhibitor of BACE1 is a bispecific antibody, e.g., with one arm targeting BACE and the other recognizing transferrin receptor to boost brain penetrance, or a camelid antibody that bind and inhibit BACE1.

11. The method of claims 1-5, wherein the oligonucleotide is 15 to 21 nucleotides in length.

12. The method of claims 1-5, wherein at least one nucleotide of the oligonucleotide is a nucleotide analogue.

13. The method of claims 1-5, wherein at least one nucleotide of the oligonucleotide comprises a 2′ O-methyl.

14. The method of claims 1-5, wherein the oligonucleotide comprises at least one ribonucleotide, at least one deoxyribonucleotide, or at least one bridged nucleotide.

15. The method of claims 1-5, wherein the bridged nucleotide is a LNA nucleotide, a cEt nucleotide or a ENA modified nucleotide.

16. The method of claims 1-5, wherein each nucleotide of the oligonucleotide is a LNA nucleotide.

17. The method of claims 1-5, wherein one or more of the nucleotides of the oligonucleotide comprise 2′-fluoro-deoxyribonucleotides, 2′-O-methyl nucleotides, ENA nucleotide analogues, or LNA nucleotides.

18. The method of claims 1-5, wherein the nucleotides of the oligonucleotide comprise phosphorothioate internucleotide linkages between at least two nucleotides.

19. The method of claims 1-5, wherein the oligonucleotide is a gapmer or a mixmer.
